SEQUENCE LISTING (24 sequences)

SEQ ID NO: 1 - protein, 231 aa, Bacillus cereus
MMGYKAMLFDLDDTLLDRDKAVEALFLIVLEKCYENVDGAAKSNMLQKFKEYDKREYGISNKTTVLESLFDEFTPRYRLPRNYIQDFWNNNFPRCFSIDQNTIHFLNQIKKHCKVGIITNGSTQRQKAKIFNTNLNKYFETIIISEEVGFSKPDKRIFELALNKLNLQPENTLFVGDDLEKDIAGPQNANIKGVWFNPQKIKNTTKIQPYAEINTLDSLLSYVTPQYFYNK

SEQ ID NO: 2 - DNA, 696 bp, Bacillus cereus
atgatgggttataaagcgatgctgtttgatttagatgatacattacttgatagggataaagcagtagaggcattatttttaattgttttagaaaagtgttatgaaaatgtagatggtgcggctaaaagcaacatgttacagaaattcaaagaatacgataaaagagaatatggtataagtaataaaacgacagttttagaatcattgtttgatgaattcacgccaaggtatagattgccacgcaattacatccaagatttttggaataataatttccctagatgtttttcaatagaccaaaatactattcatttcttaaatcaaataaagaagcactgtaaagttggaattataacaaatggctcaactcagaggcaaaaagctaaaatatttaacacgaatttaaataagtattttgaaacaatcattatttctgaagaagtgggatttagtaaacctgataaacgtatattcgaactagcattaaataagttaaatttacaaccggaaaatactttattcgttggggatgacttagaaaaggatattgctggtcctcaaaatgcaaatataaaaggtgtgtggtttaaccctcagaaaatcaagaatactaccaaaatacaaccatatgccgagattaacactttggatagtttgttaagttatgttactccacaatatttttataacaagtaa

SEQ ID NO: 3 - protein, 236 aa, Bacillus cereus
MKYKVILFDVDDTLLDFPETERHALHNAFVQFDMPTGYNDYLASYKEISNGLWRDLENKMITLSELAVDRFRQLFALHNIDVDAQQFSDVYLENLGKEVHLIEGAVQLCENLQDCKLGIITNGYTKVQQSRIGNSPLCNFFDHIIISEEVGHQKPAREIFDYAFEKFGITDKSSVLMVGDSLTSDMKGGEDYGIDTCWYNPSLKENGTDVNPTYEVESLLQILEIVEVAEEKVASF

SEQ ID NO: 4 - DNA, 711 bp, Bacillus cereus
atgaaatacaaagttatattattcgacgtagatgatacattattagatttccctgaaacggaaagacacgcattacataatgcgtttgtacagtttgatatgcctacagggtataatgattatcttgcaagctataaagagattagtaatggattatggagagatttagaaaataaaatgattacgctaagtgaattagcagtagatcgatttagacaattatttgcacttcataatatagacgtagatgcacagcaatttagtgatgtataccttgaaaatttagggaaggaagtacatcttatagaaggcgcagtacaattatgtgaaaatctacaagattgcaagttaggtattattacgaatggatatacgaaggtgcaacaatcaagaatcggaaattcacctttatgtaatttctttgatcacattattatttctgaagaagttggtcatcaaaaaccagcacgtgagatttttgattatgcgtttgagaagtttgggattactgataaatcaagcgtactaatggttggagattcgttaacttctgatatgaaaggcggagaagattacggcattgatacgtgttggtataatccgagtttgaaagaaaacgggacagatgttaacccgacttatgaagtggagagtctgctccaaattttagaaattgtagaagtggcggaagaaaaggtagcttcattttaa

SEQ ID NO: 5 - protein, 231 aa, Bacillus cereus
MKKYKTLLFDVDDTLLDFQKAEKVALRVLFEEKGIPLTDEIEARYKKINKGLWDAFEKGELSRNEVVNTRFSLLFKEYGEEVNGILFENNYRNYLEEGNQLMQGAFEFINQIQGEYELYIVTNGVSKTQDKRLRNAGLHSLFKDVFVSEDTGFQKPMKEYFDYVFERIPNFAPEEGLIIGDSLSADIKGGYVAGIDTCWFNPERKLNDSGIIPTYEVHNFEELEALLKQHV

SEQ ID NO: 6 - DNA, 696 bp, Bacillus cereus
atgaaaaaatataaaacattgctatttgatgtagatgatacattattagatttccaaaaggctgaaaaagtggctttacgggtgctttttgaagagaagggaatccctttaacagacgagatagaggctcgttataaaaagataaataaaggtctttgggatgcttttgaaaaaggtgaactatcacgcaatgaagttgtaaatacacgattctctctgttgtttaaagagtatggagaagaagtaaatggaatattattcgaaaataattatcgtaactacttagaagaaggaaatcaactcatgcaaggtgcatttgaatttataaatcaaattcaaggggagtatgaattgtatatagtgacaaatggtgtttctaagacgcaagataaacgtttgcgcaatgcagggttacattcattatttaaagatgtctttgtttcggaagatacaggttttcaaaaaccgatgaaagagtattttgattatgtttttgaacggattcctaactttgcacctgaagaagggctgattattggggattcattaagcgctgatattaaaggtggatatgtagcgggaattgatacttgttggtttaatccagaaaggaaattaaatgatagtgggattataccgacatatgaagtgcataattttgaggagttagaagcgttattgaagcagcatgtgtaa

SEQ ID NO: 7 - protein, 247 aa, Bacillus megaterium
MQKQTLLFNLDDTLVHCNKYFRDTINAFVAQLQEWFENLTKEEIKQKQLEIDLKSIEKHGLHSSRFPESLVATYLFFSEQNHQDIHEDRVQRVKKIGQSVFEVPIEPLPNMYEVLDKLKEQGHDLYLHTGGDEENQGRKIVQLELAKYFEKRVFISEQKDTAALKEILNQIKYDPKKTWMIGNSLKTDIRPAVEAGIHAIHVPSELEWSYNQVKIDVAPKGKLITVNSLLEVPEIIQKHAQETVVST

SEQ ID NO: 8 - DNA, 744 bp, Bacillus megaterium
atgcagaagcaaaccctactttttaatttagacgatacgcttgttcactgtaataaatattttagagacaccataaatgcttttgtggctcaattgcaggaatggtttgagaatctgactaaggaagaaataaaacaaaagcagttagagatagatttaaaaagtatcgaaaagcatggccttcactcttcccgatttccagaatcattagtagctacttatttattttttagtgaacaaaatcatcaagatatccatgaagatagagtccaaagagtcaaaaaaattggtcaaagtgtatttgaagtaccgatagagcctttgccaaatatgtatgaagtattagataagcttaaagaacaaggacatgatctctacttacacacaggtggagacgaggaaaatcaaggtagaaaaattgttcagctagagctagctaaatactttgaaaaaagggtatttatttctgaacaaaaagatacagctgcattaaaagaaatactaaaccaaattaagtacgatccaaaaaagacttggatgattggcaattcattaaaaaccgatataagaccggcagtagaagctggaatacatgcgattcacgtaccttctgaactggagtggagctacaaccaagttaaaattgatgttgcgccaaaaggtaaattgataaccgttaattcgttgctggaagttccagaaatcattcaaaagcatgctcaagaaacggttgtgtccacctaa

SEQ ID NO: 9 - protein, 219 aa, Bacillus megaterium
MKFKTILFDAYGTLFDVHTVIETCDKLYPERGESISHLWRLKQIEYAMQYQLMGRYVDFYTLTNQSLKYAAEANDIDLTEHEEKMLMSAYMKLNVYDEVKEVLAYLKEKGYRLAIFTNGPKHMIDPLVSYHNMNHLFEDVISVDEIKQYKPTMASYHYAKNKMNAKREEVLFLSSNTWDIAGAKNYGFATAWVNRKGNVAEHKELQPNVIIKSLNQLIK

SEQ ID NO: 10 - DNA, 660 bp, Bacillus megaterium
atgaaatttaaaacgattttatttgatgcatatggaacgctgtttgatgtacataccgttattgaaacgtgtgataagctgtatccggagcggggagaaagtattagccatttatggcgtttgaagcaaattgaatatgcaatgcaatatcagctgatgggacgatacgttgacttttatacattaacaaatcaatccctaaaatatgcagcagaagcaaatgatattgatttaacggaacatgaagaaaaaatgctgatgagtgcttacatgaagctgaatgtctacgatgaggtcaaagaggtgctcgcctatttaaaagaaaaaggatatcgtctggctatttttacgaatggtcctaagcacatgattgatccgcttgtttcttatcacaacatgaatcatttatttgaagacgttatttccgtagacgaaatcaaacagtacaagcctacgatggcaagctatcactatgcaaaaaataaaatgaacgcaaaaagagaagaagtattatttttgtcatcgaatacatgggatattgccggtgctaaaaactacgggttcgcaacggcttgggtcaaccgaaaaggaaatgtagctgaacataaagaattgcagccaaacgtaatcattaaaagcttaaatcagcttattaaataa

SEQ ID NO: 11 - protein, 238 aa, Bacillus megaterium
MKTYRTLLFDIDNTLLDFNAAEEQALQLLFANHDIPLTEESKKRYSLINQGLWTAFEENKISREQVVNTRFSTLCKEYGIEKDGKLLEAEYRTYLNNGHQLIDGAFEVIKNLSCHYELYVVTNGVSATQYKRLQDSGLYPYFKEVFVSEDTGFQKPMKEYFDYVFSRITELSVHETLIIGDSLSADIQGGQLAGIDTCWFNPQKKKNDTNWNSTHEIGRLQELYILLNVNNEVASVQV

SEQ ID NO: 12 - DNA, 717 bp, Bacillus megaterium
atgaaaacataccgaacgcttctatttgacattgataatacgcttttagattttaacgcagccgaagagcaggctttacagcttctttttgctaaccacgatattccgttgactgaggaaagcaaaaaacgttatagtttgataaaccaggggttatggacagcttttgaagaaaataaaataagccgtgagcaagttgtaaatactcgcttttctactctatgtaaagaatatggaatagaaaaagacggaaagctacttgaagcagagtaccgaacgtatttgaacaatggccatcagctaattgacggagcatttgaggtaattaaaaacctgagctgtcactatgagttatatgttgttacaaatggcgtatcggctacgcaatataaacgccttcaggactcaggtctatatccttattttaaagaagtatttgtttcagaagatacgggctttcaaaagcctatgaaggaatattttgattatgttttttcacgtataacagagttgtccgtacatgaaacattaattattggagattctttgagtgcagacattcaaggcgggcagctagccggtattgatacgtgttggtttaatccgcagaaaaaaaagaatgatacaaattggaattctacccatgaaataggaaggcttcaagagctttatattctgctgaacgtaaataatgaagtagcttcggtacaagtgtaa

SEQ ID NO: 13 - DNA, 38 bp, Artificial Sequence
  primer
aagaaggagatataccatgatgggttataaagcgatgc

SEQ ID NO: 14 - DNA, 41 bp, Artificial Sequence
  primer
gcattatgcggccgcaagctttacttgttataaaaatattg

SEQ ID NO: 15 - DNA, 40 bp, Artificial Sequence
  primer
aagaaggagatataccatgaaatacaaagttatattattc

SEQ ID NO: 16 - DNA, 41 bp, Artificial Sequence
  primer
gcattatgcggccgcaagctttaaaatgaagctaccttttc

SEQ ID NO: 17 - DNA, 38 bp, Artificial Sequence
  primer
aagaaggagatataccatgaaaaaatataaaacattgc

SEQ ID NO: 18 - DNA, 40 bp, Artificial Sequence
  primer
gcattatgcggccgcaagctttacacatgctgcttcaata

SEQ ID NO: 19 - DNA, 37 bp, Artificial Sequence
  primer
aagaaggagatataccatgcagaagcaaaccctactt

SEQ ID NO: 20 - DNA, 41 bp, Artificial Sequence
  primer
gtggtggtggtggtgctcgattaggtggacacaaccgtttc

SEQ ID NO: 21 - DNA, 37 bp, Artificial Sequence
  primer
aagaaggagatataccatgaaatttaaaacgatttta

SEQ ID NO: 22 - DNA, 40 bp, Artificial Sequence
  primer
gtggtggtggtggtgctcgattatttaataagctgattta

SEQ ID NO: 23 - DNA, 36 bp, Artificial Sequence
  primer
aagaaggagatataccatgaaaacataccgaacgct

SEQ ID NO: 24 - DNA, 41 bp, Artificial Sequence
  primer
gtggtggtggtggtgctcgattacacttgtaccgaagctac

(19) United States
(12) Patent Application Publication
(10) Pub. No.: US 2018/0104647 A1
(43) Pub. Date: Apr. 19, 2018

(54) RECOMBINANT MICROORGANISM INCLUDING GENETIC MODIFICATION THAT INCREASES 2-HALOACID DEHALOGENASE ACTIVITY AND METHOD OF REDUCING CONCENTRATION OF FLUORINATED METHANE IN SAMPLE BY USING THE SAME

(71) Applicant: Samsung Electronics Co., Ltd, Suwon-si (KR)

(72) Inventors: Jinsuk Lee, Seoul (KR); Yukyung Jung, Hwaseong-si (KR); Jinhwan Park, Suwon-si (KR)

(21) Appl. No.: 15/701,217

(22) Filed: Sep. 11, 2017

(30) Foreign Application Priority Data
Oct. 17, 2016 (KR) 10-2016-0134549

(51) Int. Cl. B01D 53/84; C12N 9/14; C12N 15/70; B01D 53/70

(52) U.S. Cl. CPC B01D 53/84; C12N 9/14; C12Y 308/01002; B01D 2257/2066; B01D 53/70; B01D 2251/95; C12N 15/70

(57) ABSTRACT

Provided is a recombinant microorganism including a genetic modification that increases a 2-haloacid dehalogenase activity, a method of preparing the recombinant microorganism, and a method of using the recombinant microorganism for reducing the concentration of fluorinated methane in a sample.

### CROSS-REFERENCE TO RELATED APPLICATION

This application claims the benefit of Korean Patent Application No. 10-2016-0134549, filed on Oct. 17, 2016, in the Korean Intellectual Property Office, the disclosure of which is incorporated herein in its entirety by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: One 20,286 byte ASCII (Text) file named “728866_ST25.TXT,” created Sep. 11, 2017.

### BACKGROUND

### 1. Field

The present disclosure relates to a recombinant microorganism including a genetic modification that increases a 2-haloacid dehalogenase activity, a composition for use in reducing a concentration of fluorinated methane in a sample, the composition including the recombinant microorganism, and a method of reducing the concentration of fluorinated methane in a sample.

### 2. Description of the Related Art

The emissions of greenhouse gases which have accelerated global warming are a serious environmental problems and regulations to reduce and prevent the emissions of greenhouse gases have been tightened. Among the greenhouse gases, fluorinated gases (F-gas) such as perfluorocarbons (PFCs), hydrofluorocarbons (HFCs), or sulfur hexafluoride (SF₆) show low absolute emission, but have a long half-life and a very high global warming potential, resulting in significant adverse environmental impacts. The amount of F-gas emitted from semiconductor and electronics industries, which are major causes of F-gas emission, has exceeded the assigned amount of greenhouse gas emissions and continues to increase. Therefore, costs required for degradation of greenhouse gases and greenhouse gas emission allowances are increasing every year.

A pyrolysis or catalytic thermal oxidation process has been generally used in the decomposition of F-gas. However, this process has disadvantages of limited decomposition rate, emission of secondary pollutants, high cost, etc. To solve this problem, biological decomposition of F-gas using a microbial biocatalyst has been proposed. Accordingly, there remains a need for new compositions and methods to treat F-gas in more economical and environmentally-friendly manner.

### SUMMARY

An aspect provides a recombinant microorganism including a genetic modification that increases a 2-haloacid dehalogenase (HAD) activity as compared to the same microorganism without the genetic modification, as well as a method for preparing the recombinant microorganism.

Another aspect provides a composition for use in reducing a concentration of CHₙF₄₋ₙ (where n is an integer of 0 to 3) in a sample, the composition including the recombinant microorganism including the genetic modification that increases the HAD activity.

Still another aspect provides a method of reducing a concentration of CHₙF₄₋ₙ in a sample, the method including contacting the recombinant microorganism including the genetic modification that increases the HAD activity with the sample containing CHₙF₄₋ₙ (where n is an integer of 0 to 3) to reduce the concentration of CHₙF₄₋ₙ (where n is an integer of 0 to 3) in the sample.

### BRIEF DESCRIPTION OF THE DRAWINGS

These and/or other aspects will become apparent and more readily appreciated from the following description of the embodiments, taken in conjunction with the accompanying drawings in which:

FIG. 1 shows a vector map of a pET-BC HAD vector; and

FIG. 2 shows a vector map of a pET-BG HAD vector.

### DETAILED DESCRIPTION

The terms “increase in activity” or “increased activity” or “increases activity” and the like, as used herein, may refer to a detectable increase in an activity of a cell, a protein, or an enzyme. The “increase in activity” or “increased activity” may also refer to an activity level of a modified (e.g., genetically engineered) cell, protein, or enzyme that is higher than that of a comparative cell, protein, or enzyme of the same type, such as a cell, protein, or enzyme that does not have a given genetic modification (e.g., original or “wild-type” cell, protein, or enzyme). The phrase “activity of a cell” may refer to an activity of a particular protein or enzyme of a cell. For example, an activity of a modified or engineered cell, protein, or enzyme may be increased by about 5% or more, about 10% or more, about 15% or more, about 20% or more, about 30% or more, about 50% or more, about 60% or more, about 70% or more, or about 100% or more relative to an activity of a non-engineered cell, protein, or enzyme of the same type, i.e., a wild-type cell, protein, or enzyme. A cell having an increased activity of a protein or an enzyme may be identified by using any method known in the art.

An increase in an activity of an enzyme or a polypeptide may be achieved by an increase in expression or specific activity. The increase in expression may be caused by introduction of a polynucleotide encoding the enzyme or the polypeptide into a cell; by otherwise increasing of the copy number of a gene encoding the enzyme or polypeptide, by modification of a regulatory region of the polynucleotide encoding the enzyme or polypeptide. A microorganism into which the gene is introduced may be a microorganism endogenously including the gene (e.g., the exogenous gene is homologous to the microorganism), or a microorganism that does not endogenously include the gene (e.g., the exogenous gene is heterologous to the microorganism). The gene may be operably linked to a regulatory sequence that allows expression thereof, for example, a promoter, an enhancer, a polyadenylation site, or a combination thereof. The polynucleotide whose copy number is increased may be endogenous or exogenous. An endogenous gene is a gene that exists in thegenetic material of a microorganism prior to the genetic modification that increases the activity of the enzyme or polypeptide. An exogenous gene refers to a gene that is introduced into a cell in the genetic modification, and may be homologous or heterologous with respect to a host cell into which the gene is introduced. The term “heterologous” means “foreign” or “not native.” Thus, an “exogenous” gene can be introduced despite the preexistence of the same or similar gene in the microorganism.

An “increase of the copy number” may be caused by introduction of an exogenous gene or amplification of an endogenous gene. Optionally, the increase in copy number achieved by genetically engineering a cell to introduce a gene that does not exist in a non-engineered cell. The introduction of the gene may be mediated by a vehicle such as a vector. The introduction may be via a transient introduction in which the gene is not integrated into a genome (e.g., in an episome such as a plasmid), or via integration of the gene into the genome. The introduction may be performed, for example, by introducing a vector into the cell, the vector including a polynucleotide encoding a target polypeptide, and then, replicating the vector in the cell, or by integrating the polynucleotide into the genome.

The introduction of the gene may be performed via a known method, for example, transformation, transfection, or electroporation. The gene may be introduced via a vehicle or as it is. The term “vehicle”, as used herein, refers to a nucleic acid molecule that is able to deliver other nucleic acids linked thereto. As a nucleic acid sequence mediating introduction of a specific gene, the vehicle used herein is construed to be interchangeable with a vector, a nucleic acid construct, and a cassette. The vector may include, for example, a plasmid vector, a virus-derived vector, etc. The plasmid can be a circular double-stranded DNA molecule linkable with another DNA. The vector may include, for example, a plasmid expression vector, a virus expression vector, such as a replication-defective retrovirus, adenovirus, adeno-associated virus, or a combination thereof.

The genetic modification used in the present disclosure may be performed by a molecular biological method known in the art.

The term “parent cell” refers to an original cell or a cell of the same type as an original cell from which a genetically engineered cell is produced. With respect to a particular genetic modification, the “parent cell” may be a cell (whether previously engineered or not) that lacks the particular genetic modification, but is identical in all other respects. Thus, the parent cell may be a cell that is used as a starting material to produce a genetically engineered microorganism having increased activity of a given protein (e.g., a protein having a sequence identity of about 95% or higher amino acid identity with respect to 2-haloacid dehalogenase). The same comparison is also applied to other genetic modifications. In some embodiments, the parent cell can be a wild-type cell.

The term “gene”, as used herein, refers to a nucleotide fragment expressing a particular protein, and may or may not be operably linked to a regulatory sequence (e.g, a 5′-non coding sequence and/or a 3′-non coding sequence).

The term “sequence identity” of a polynucleotide or a polypeptide, as used herein, refers to a degree of identity between bases or amino acid residues of sequences obtained after the sequences are aligned so as to best match in certain comparable regions. The sequence identity is a value that is measured by comparing two sequences in certain comparable regions via optimal alignment of the two sequences, in which portions of the sequences in the certain comparable regions may be added or deleted compared to reference sequences. A percentage of sequence identity may be calculated by, for example, comparing two optimally aligned sequences in the entire comparable regions, determining the number of locations in which the same amino acids or nucleic acids appear to obtain the number of matching locations, dividing the number of matching locations by the total number of locations in the comparable regions (that is, the size of a range), and multiplying a result of the division by 100 to obtain the percentage of the sequence identity. The percentage of the sequence identity may be determined using a known sequence comparison program, for example, BLASTN (NCBI), BLASTP (NCBI), CLC Main Workbench (CLC bio), MegAlign™ (DNASTAR Inc),), or EMBOSS Needle. For example, the Needleman-Wunsch global alignment algorithm in EMBOSS may be used with the default settings (gap opening penalty 10, gap extension penalty 0.5; end gap penalty=false, end gap open-10, for amino acid sequence comparisons, the BLOSUM62 matrix is used).

Various levels of sequence identity may be used to identify various types of polypeptides or polynucleotides having the same or similar functions or activities. For example, the sequence identity may include a sequence identity of about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, about 99% or more, or 100%.

Where a polynucleotide sequence encoding a given protein, other polynucleotide sequences can be substituted based on the degeneracy of the genetic code.

The term “genetic modification”, as used herein, refers to an artificial alteration in a constitution or structure of a genetic material of a cell.

In the present disclosure, % represents w/w %, unless otherwise mentioned.

An aspect of the disclosure provides a recombinant microorganism including a genetic modification that increases a 2-haloacid dehalogenase (HAD) activity.

2-Haloacid dehalogenase is an enzyme that catalyzes a chemical reaction of 2-haloacid+H₂O ⇔2-hydroxyacid+halide. Thus, two substrates of this enzyme are 2-haloacid and H₂O, and its two products are 2-hydroxyacid and halide. This enzyme belongs to the family of hydrolases which act on halide bonds in carbon-halide compounds. However, with regard to reducing a concentration of CHₙF₄₋ₙ (where n is an integer of 0 to 3) in a sample, the recombinant microorganism should not be construed as being limited to this particular mechanism. The HAD may belong to EC 3.8.1.2. The HAD may be exogenous or endogenous. The HAD may be selected from the group consisting of HADs derived from the genus Bacillus, Pseudomonas, Azotobacter, Agrobacterium, and Escherichia. The HAD may be derived from a strain selected from the group consisting of Bacillus cereus, Bacillus thuringiensis, and Bacillus megaterium.

The HAD may be a polypeptide having a sequence identity of about 95% or more with respect to amino acid sequences of SEQ ID NOS: 1, 3, 5, 7, 9, and 11.

With regard to the above microorganism, the genetic modification may increase expression of a gene encoding the HAD. The genetic modification may increase the copy number of the HAD gene. The genetic modification may increase the copy number of one or more genes among genes encoding polypeptides having a sequence identity of 95% or more with respect to the amino acid sequences of SEQ ID NOS: 1, 3, 5, 7, 9, and 11, respectively. The genes may have a sequence identity of 95% or more with respect to the nucleotide sequences of SEQ ID NOS: 2, 4, 6, 8, 10, and 12, respectively. The genetic modification may be the introduction an exogenous gene encoding HAD, for example, via a vehicle such as a vector. The gene encoding HAD may exist within or outside the chromosome. The introduced gene encoding HAD may be a plurality of genes (e.g., replicates of the same gene and/or a mixture of genes encoding multiple gene variants), for example, 2 or more, 5 or more, 10 or more, 50 or more, 100 or more, or 1000 or more.

The microorganism may belong to the genus Escherichia, Bacillus, Xanthobacter, Pseudomonas, Azotobacter, or Agrobacterium. The microorganism may be, for example, of the species E. coli or X. autotrophicus.

The microorganism may further include one or more genes selected from the group consisting of a gene encoding ArgU and a gene encoding ProL. The gene encoding ArgU may encode tRNA that recognizes the arginine codons, AGA and AGG. The gene encoding ProL may encode tRNA that recognizes the proline codon, CCC. The microorganism may be BL21-CodonPlus(DE3)-RP (or another strain having similar codon bias reducing features) which further includes both of the gene encoding ArgU and the gene encoding ProL.

The microorganism may further include, or be modified to include, one or more genes selected from the group consisting of the gene encoding ArgU, a gene encoding ileY, and a gene encoding ileW. The gene encoding ileY may encode tRNA that recognizes the isoleucine codon, AUA. The gene encoding ileW may encode tRNA that recognizes the leucine codon, CUA. The microorganism may be BL21-CodonPlus-RIL or BL21-CodonPlus(DE3)-RIL which further includes all of the genes encoding ArgU, the gene encoding ileY, and the gene encoding ileW.

The recombinant microorganism may reduce a concentration of CHₙF₄₋ₙ (where n is an integer of 0 to 3) (referred to herein as “fluorinated methane”) in a sample. The reducing may be performed by introducing a hydroxyl group to carbon of the fluorinated methane by action of the HAD enzyme protein on C—F or C—H bond thereof or by accumulating the fluorinated methane inside the cell of the microorganism. Further, the reducing may include cleaving of C—F bonds of CHₙF₄₋ₙ, converting of CHₙF₄₋ₙ into other materials, or intracellular accumulating of CHₙF₄₋ₙ. The sample may be in a liquid or gas state. The sample may be industrial waste water or waste gas. The sample may be any sample including the fluorinated methane. The fluorinated methane may include CF₄, CHF₃, CH₂F₂, CH₃F, or a mixture thereof.

Reducing CHₙF₄₋ₙ in the sample encompases a reduction in CHₙF₄₋ₙ to any degree, including complete removal of CHₙF₄₋ₙ. The sample may be a gas or a liquid. The composition may further include a material that increases solubility of the fluorinated methane for a medium or a culture.

Another aspect provides a composition used for reducing a concentration of CHₙF₄₋ₙ (where n is an integer of 0 to 3) in a sample, the composition including the recombinant microorganism including the genetic modification that increases 2-haloacid dehalogenase (HAD) activity. Still another aspect provides a method of reducing a concentration of CHₙF₄₋ₙ in a sample, the method including contacting the recombinant microorganism with the sample containing CHₙF₄₋ₙ (where n is an integer of 0 to 3) to reduce the concentration of CHₙF₄₋ₙ (where n is an integer of 0 to 3) in the sample, in which the recombinant microorganism includes the genetic modification that increases 2-haloacid dehalogenase (HAD) activity. The recombinant microorganism, sample and fluorinated methane are the same as described above.

The composition or method may reduce the concentration of fluorinated methane in the sample by contacting the composition with the sample. The contacting may be performed in a liquid or solid phase. The contacting may be performed, for example, by contacting a culture of the cultured microorganism with the sample during culturing. Optionally, the culturing may be performed under conditions where the microorganism may proliferate. In some embodiments, the contacting may be performed in a sealed container (e.g., one that prevents the escape of fluorinated gas). The phrase “sealed container” represents an air tight condition. The contacting may be performed in a closed, flow-through type system, permitting continuous flow of gas. The contacting may include culturing or incubating the recombinant microorganism while contacting it with a sample containing CHₙF₄₋ₙ (where n is an integer of 0 to 3). The contacting may include culturing the recombinant microorganism under conditions where the recombinant microorganism may survive or proliferate, or conditions where the recombinant microorganism may be allowed to be in a resting state. The contacting may be performed when the growth stage of the microorganism is in an exponential phase or a stationary phase. The culturing may be performed under aerobic or anaerobic conditions.

The sample may be in a liquid or gas state. The sample may be industrial waste water or waste gas. The sample may include passive contacting of the sample with the culture of the microorganism and active contacting of the sample with the culture of the microorganism. The sample may be, for example, sparged into the culture of the microorganism. That is, the sample may be sparged into a medium or culture. The sparging may be sparging of the sample from the bottom to the top of the medium or culture. The sparging may be via injecting of droplets or bubbles of the sample.

The contacting of the recombinant microorganism with the sample may be performed in a batch or continuous manner. The contacting may include, for example, contacting a fresh recombinant microorganism including the genetic modification that increases HAD activity with a sample obtained in a previous contacting step. In otherwords, the method can comprise repeatedly contacting the sample with recombinant microorganisms, such that the sample is contacted with fresh microorganisms twice or more, for example, twice, three times, five times, or ten times or more. Alternately or in addition, fresh microorganism may be contacted with the sample by continuous infusion of fresh microorganism and, optionally, continuous removal of used microorgansim. The term “fresh” microorganism means a recombinant microorganism that has not yet been contacted with the sample. “Used” microorganism means a recombinant microorganism that has been contacted with the sample. The contacting may be continued or repeated until the concentration of fluorinated methane in the sample reaches a desired reduced concentration.

Still another aspect provides a method of producing the microorganism, the method including introducing into the microorganism the genetic modification that increases 2-haloacid dehalogenase (HAD) activity. The method may be a method of producing the microorganism, including introducing the gene encoding HAD into the microorganism. The introducing of the gene encoding HAD may be achieved by introducing of a vehicle including an exogenous gene into the microorganism. With regard to the method, the genetic modification may include amplification of the gene, manipulation of the regulatory sequence of the gene, or manipulation of the sequence of the gene itself. The manipulation may be insertion, substitution, conversion, or addition of nucleotides.

The method may further include increasing the copy number of one or more genes selected from the group consisting of the gene encoding ArgU and the gene encoding ProL in the microorganism. The method may further include increasing the copy number of one or more genes selected from the group consisting of the gene encoding ArgU, the gene encoding ileY, and the gene encoding ileW in the microorganism.

The recombinant microorganism according to an aspect may be used to remove CHₙF₄₋ₙ in the sample.

The composition according to another aspect may be used to remove CHₙF₄₋ₙ in the sample.

The method of reducing the concentration of CHₙF₄₋ₙ in the sample according to still another aspect may be used to efficiently reduce the concentration of CHₙF₄₋ₙ in the sample.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. These Examples are provided for illustrative purposes only, and the invention is not intended to be limited by these Examples.

### Example 1: Decomposition of CF₄ by Bacillus Strain

Bacillus cereus (KCTC 3624) and Bacillus megaterium (DSM 32) strains were cultured in LB medium under shaking at 30° C. and 230 rpm until cell density reached OD₆₀₀=3.0. 10 ml of each cell culture was added to a 60 ml-serum bottle, and the bottles were sealed. The LB medium included 10 g of tryptone, 5 g of yeast extract, and 10 g of NaCl per 1 L of distilled water.

Next, gas-phase CF₄ was injected through a rubber stopper of a cap of the serum bottle using a syringe to a headspace concentration of 1000 ppm. Thereafter, the serum bottles were incubated for 5 days under shaking at 30° C. and 230 rpm. Each experiment was performed in triplicate.

After incubation, 0.5 ml of the headspace gas containing no medium in the serum bottle was collected using a 1.0 ml-headspace syringe and injected into GC (Agilent 7890, Palo Alto, Calif., USA). The injected headpace sample was separated through a CP-PoraBOND Q column (25 m length, 0.32 mm i.d., 5 um film thickness, Agilent), and changes in the CF₄ concentration were analyzed by mass spectrometry (Agilent 5973, Palo Alto, Calif., USA). As a carrier gas, helium was used, and applied to the column at a flow rate of 1.5 ml/min. Gas chromatography (GC) conditions were as follows: The inlet temperature was 250° C., initial temperature was maintained at 40° C. for 2 minutes then raised to 290° C. at a rate of 20° C./min. Mass spectrometry conditions were as follows: Ionization energy was 70 eV, interface temperature was 280° C., ion source temperature was 230° C., and quadrupole temperature was 150° C.

Table 1 shows percentages of residual CF₄ in the samples when B. cereus and B. megaterium strains were cultured under the conditions as above. As shown in Table 1, B. cereus and B. megaterium strain cultures showed about 6.63% and about 9.33% decreases in the headspace concentrations of CF₄, compared to a control group containing only LB medium, respectively.

TABLE 1

| Strain | Residual CF₄(%) |
|---|---|
| Control (medium) | 100.00 |
| B. cereus | 93.37 |
| B. megaterium | 90.67 |

### Example 2: Preparation of Recombinant E. coli Introduced with Bacillus cereus-Derived HAD Gene and Decomposition of CF₄ by Using the Same

1. Amplification of Bacillus cereus-Derived HAD Gene (BC HAD) and Introduction of the Gene into E. coli

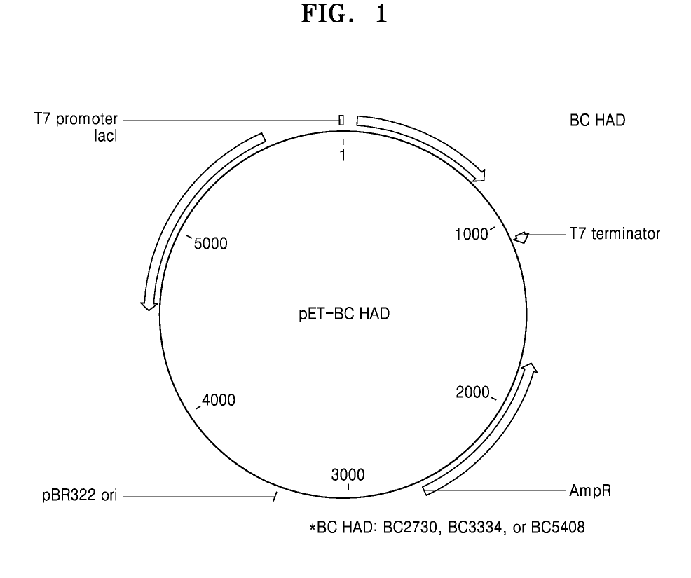

B. cereus (KCTC 3624) was cultured in an LB medium at 30° C. under shaking at 230 rpm overnight, and then genomic DNA was isolated using the total DNA extraction kit (Invitrogen Biotechnology). PCR was performed using this genomic DNA as a template and a set of primers having nucleotide sequences given in Table 2 to amplify and obtain BC2730, BC3334, and BC5408 genes, respectively. The BC HAD genes thus amplified were ligated with pETDuet-1 (Novagen, Cat. No. 71146-3), respectively which was digested with restriction enzymes, Ncol and HindIII, using the InFusion Cloning Kit (Clontech Laboratories, Inc.) to prepare 3 kinds of pET-BC HAD vectors. FIG. 1 shows a vector map of the pET-BC HAD vector. BC2730, BC3334, and BC5408 have amino acid sequences of SEQ ID NOS: 1, 3, and 5, respectively. Their genes have nucleotide sequences of SEQ ID NOS: 2, 4, and 6, respectively.

Next, each of the prepared three kinds of pET-BC HAD vectors, pET-BC2730, pET-BC3334, and pET-BC5408 was introduced to E. coli BL21 by a heat shock method, and then cultured on a LB plate containing 100 μg/mL ampicillin. Strains showing ampicillin resistance were selected. Finally, three strains thus selected were designated as a recombinant E. coli BL21/pET-BC2730, BL21/pET-BC3334, and BL21/pET-BC5408, respectively.

TABLE 2

| BC HAD gene | Primer sequence (SEQ ID NO) |
|---|---|
| BC2730 | Forward: SEQ ID NO: 13 |
| | Reverse: SEQ ID NO: 14 |
| BC3334 | Forward: SEQ ID NO: 15 |
| | Reverse: SEQ ID NO: 16 |
| BC5408 | Forward: SEQ ID NO: 17 |
| | Reverse: SEQ ID NO: 18 |

2. Decomposition of Perfluoromethane by BC HAD Gene-Introduced E. coli

In this section, it was examined whether the three kinds of recombinant E. coli BL21/pET-BC HAD strains prepared in section (1) affect removal of CF₄ in a sample.

In detail, each of BL21/pET-BC2730, BL21/pET-BC3334, and BL21/pET-BC5408 was cultured in a TB medium at 30° C. under shaking at 230 rpm. At OD₆₀₀ of about 0.5, 0.2 mM of IPTG was added thereto, followed by culturing at 20° C. under shaking 230 rpm overnight. Each of the cells was harvested and suspended in an LB medium to a cell density of OD₆₀₀ of 3.0. 10 ml of each cell suspension was added to a 60 ml-serum bottle, and then the bottles were sealed. The LB medium has the same composition as in Example 1.

Next, gas-phase CF₄ was injected through a rubber stopper of a cap of the serum bottle using a syringe to its headspace concentration of 1000 ppm. Thereafter, the serum bottle was incubated for 4 days under shaking at 30° C. and 230 rpm. Each experiment was performed in triplicate. After incubation, a headspace concentration of CF₄ in the serum bottle was analyzed under the same conditions as in Example 1.

Table 3 shows percentages of residual CF₄ in the samples when the recombinant E. coli BL21/pET-BC HAD strains were cultured under the conditions as above. As shown in Table 3, the recombinant E. coli strains introduced with BC HAD (BC2730, BC3334, or BC5408) showed about 1.69% decrease, about 5.67% decrease, and about 5.44% decrease in the headspace concentrations of CF₄, compared to a control group introduced with an empty vector.

TABLE 3

| Recombinant strain | Residual CF₄(%) |
|---|---|
| Control (empty vector) | 100.00 |
| BC2730 | 98.31 |
| BC3334 | 94.33 |
| BC5408 | 94.56 |

### Example 3: Preparation of Recombinant E. coli Introduced with Bacillus megaterium-Derived HAD Gene and Decomposition of CF₄ by Using the Same

1. Amplification of Bacillus megaterium-Derived HAD Gene (BG HAD) and Introduction of the Gene into E. coli

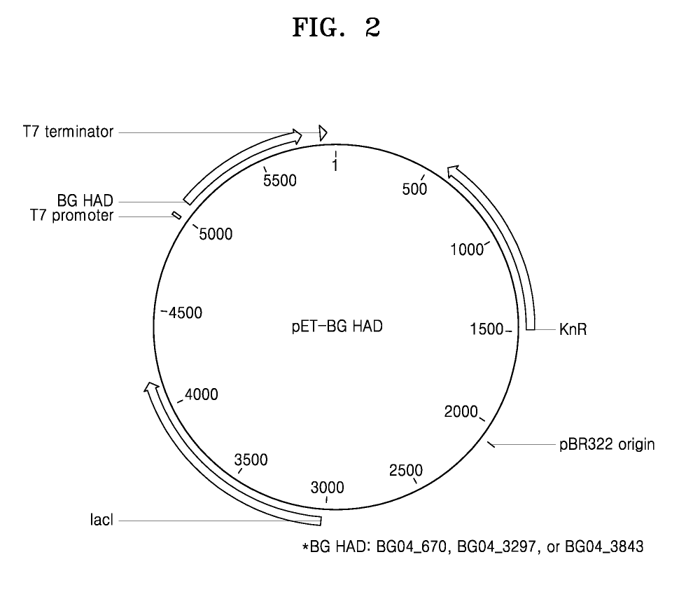

B. megaterium (DSM 32) was cultured in LB medium at 30° C. under shaking at 230 rpm overnight, and then genomic DNA was isolated using the total DNA extraction kit (Invitrogen Biotechnology). PCR was performed using this genomic DNA as a template and a set of primers having nucleotide sequences given in Table 4 to amplify and obtain BG_04_670 BG_04_3297 and BG_04_3843 genes, respectively. BG_04_670 BG_04_3297, and BG_04_3843 have amino acid sequences of SEQ ID NOS: 7, 9, and 11, respectively. Their genes have nucleotide sequences of SEQ ID NOS: 8, 10, and 12, respectively. The BG HAD genes thus amplified were ligated with pET28a (Novagen, Cat. No. 69864-3), respectively which was digested with restriction enzymes, Ncol and XhoI, using the InFusion Cloning Kit (Clontech Laboratories, Inc.) to prepare 3 kinds of pET-BG HAD vectors. FIG. 2 shows a vector map of the pET-BG HAD vector.

Next, each of the prepared three kinds of pET-BG HAD vectors, pET-BG_04_670, pET-BG_04_3297, pET-BG_04_3843 was introduced to E. coli BL21 by a heat shock method, and then cultured on a LB plate containing 50 μg/mL kanamycin. Strains showing kanamycin resistance were selected. Finally, three strains thus selected were designated as a recombinant E. coli BL21/pET-BG_04_670, BL21/pET-BG_04_3297, and BL21/pET-BG_04_3843, respectively.

TABLE 4

| BG HAD gene | Primer sequence (SEQ ID NO) |
|---|---|
| BG04_670 | Forward: SEQ ID NO: 19 |
| | Reverse: SEQ ID NO: 20 |
| BG04_3297 | Forward: SEQ ID NO: 21 |
| | Reverse: SEQ ID NO: 22 |
| BG04_3843 | Forward: SEQ ID NO: 23 |
| | Reverse: SEQ ID NO: 24 |

Decomposition of Perfluoromethane by BG HAD Gene-Introduced E. coli

In this section, it was examined whether the three kinds of recombinant E. coli BL21/pET-BG HAD strains prepared in section (1) effect removal of CF₄ in a sample.

In detail, each of E. coli BL21/pET-BG_04_670, BL21/pET-BG_04_3297, and BL21/pET-BG_04_5408 strains was cultured in a TB medium at 30° C. under shaking at 230 rpm. At OD₆₀₀ of about 0.5, 0.2 mM of IPTG was added thereto, followed by culturing at 20° C. under stirring 230 rpm overnight. Each of the cells was harvested and suspended in LB medium to a cell density of OD₆₀₀ of 3.0. 10 ml of each cell suspension was added to a 60 ml-serum bottle, and then the bottles were sealed. The LB medium has the same composition as in Example 1.

Next, gas-phase CF₄ was injected through a rubber stopper of a cap of the serum bottle using a syringe to its headspace concentration of 1000 ppm. Thereafter, the serum bottle was incubated for 4 days under shaking at 30° C. and 230 rpm. Each experiment was performed in triplicate. After incubation, a headspace concentration of CF₄ in the serum bottle was analyzed under the same conditions as in Example 1.

Table 5 shows percentages of residual CF₄ in the samples when the recombinant E. coli BL21/pET-BG HAD strains were cultured under the conditions as above. As shown in Table 5, the recombinant E. coli strains introduced with BG HAD (BG_04_670, BG_04_3297, or BG_04_3843) showed about 6.01% decrease, about 8.55% decrease, and about 8.95% decrease in the headspace concentrations of CF₄, compared to a control group introduced with an empty vector.

TABLE 5

| Recombinant strain | Residual CF₄(%) |
|---|---|
| Control (empty vector) | 100.00 |
| BG04_670 | 93.99 |
| BG04_3297 | 91.45 |
| BG04_3843 | 91.05 |

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variation thereof is encompassed by the invention unless otherwise indicated herein or otherwise clearly contradicted by context.

What is claimed is:

1. A recombinant microorganism comprising a genetic modification that increases a 2-haloacid dehalogenase (HAD) activity as compared to a microorganism of the same type without the genetic modification.

2. The recombinant microorganism of claim 1, wherein the genetic modification increases the copy number of a gene encoding the HAD, or the genetic modification is the introduction of an exogenous gene encoding HAD.

3. The recombinant microorganism of claim 1, wherein the HAD is from Bacillus, Pseudomonas, Azotobacter, Agrobacterium, or Escherichia.

4. The recombinant microorganism of claim 1, wherein the HAD is from Bacillus cereus, Bacillus thuringiensis, and Bacillus megaterium.

5. The recombinant microorganism of claim 1, wherein the HAD belongs to EC 3.8.1.2.

6. The recombinant microorganism of claim 1, wherein the HAD is a polypeptide having a sequence identity of 95% or more with respect to an amino acid sequence of SEQ ID NO: 1, 2, 3, 4, 5, or 6.

7. The recombinant microorganism of claim 2, wherein the gene has a sequence identity of 95% or more with respect to a nucleotide sequence of SEQ ID NO: SEQ ID NO: 7, 8, 9, 10, 11, or 12.

8. The recombinant microorganism of claim 1, wherein the microorganism belongs to the genus Escherichia, Bacillus, or Xanthobacter.

9. The recombinant microorganism of claim 2, wherein the exogenous gene encoding the HAD is heterologous to the microorganism.

10. A method of reducing a concentration of CHₙF₄₋ₙ in a sample, the method comprising:
contacting the recombinant microorganism of claim 1 with a sample containing CHₙF₄₋ₙ (where n is an integer of 0 to 3) to reduce the concentration of CHₙF₄₋ₙ (where n is an integer of 0 to 3) in the sample.

11. The method of claim 10, wherein the genetic modification increases the copy number of a gene encoding the HAD.

12. The method of claim 10, wherein the HAD belongs to EC 3.8.1.2.

13. The method of claim 10, wherein the recombinant microorganism is contacted with the sample in a sealed container.

14. The method of claim 10, wherein contacting of the recombinant microorganism with the sample comprises culturing or incubating the recombinant microorganism while in contact with the sample containing CHₙF₄₋ₙ (where n is an integer of 0 to 3).

15. The method of claim 13, wherein the method comprises culturing the recombinant microorganism in the sealed container under conditions in which the recombinant microorganism proliferates.

16. A method of preparing a recombinant microorganism of claim 1, which method comprises introducing into a microorganism an exogenous gene encoding an HAD.

17. The method of claim 16, wherein the exogenous gene encoding an HAD is heterologous to the microorganism.
